(19) United States
(12) Patent Application Publication
(10) Pub. No.: US 2021/0339052 A1
(43) Pub. Date: Nov. 4, 2021

(54) Removable High Intensity Focused Ultrasound Transducer Assembly and Coupling Mechanism

(71) Applicant: Valam Corporation, Ridgefield, CT (US)

(72) Inventors: Ralf Seip, Charlotte, NC (US); Mark Carol, Cornelius, NC (US); Ron Hadani, Herzeliya (IL); Yosef Krespi, New York, NY (US); Grant Adam Morris, Frankfort, IN (US); Joshua Huff, Indianapolis, IN (US)

(21) Appl. No.: 17/197,016

(22) Filed: Mar. 9, 2021

Related U.S. Application Data
(60) Provisional application No. 62/987,868, filed on Mar. 10, 2020.

(51) Int. Cl. A61N 7/00; A61B 90/00; A61B 17/32

(52) U.S. Cl. CPC A61N 7/00; A61B 90/37; A61B 17/320068; A61B 2017/320069; A61N 2007/0052; A61B 2090/378; A61N 2007/0043

(57) ABSTRACT

There is presented a removable high-intensity focused ultrasound (HIFU) transducer and supporting system that allows for HIFU transducer assemblies and other therapeutic ultrasound assemblies to be attached to third party positioning, imaging, or guidance systems while maintaining the required focal-zone positioning ability and the required acoustic energy coupling to tissue, to mechanically or automatically ablate a target volume in unimpeded fashion. There is provided an acoustically transparent and pliable vessel that has an open top end and filled with acoustic coupling fluid, capable of holding the transducer assembly and distal end of the positioning system within it as it executes the HIFU treatment plan/process. The vessel is held in place using a transducer/positioning system and independent external fixturing mechanism.

### CROSS REFERENCE TO RELATED APPLICATIONS

This application claims benefit of and priority to U.S. Provisional Application Ser. No. 62/987,868 filed on Mar. 10, 2020, which is incorporated herein in its entirety.

### BACKGROUND

Designing application-specific therapeutic ultrasound probes is time-consuming and expensive. Each probe typically not only needs its therapeutic ultrasound transducer, but also needs a transducer-to-tissue coupling mechanism, and a transducer positioning system (manual or machine-controlled) that positions the transducer at the correct orientation and location to the tissue targeted for treatment, and mechanically or electronically translates the transducer's acoustic beam to execute a treatment plan/process. In addition, a guidance mechanism (optical, acoustic, other) is also typically required, in order to verify that the ultrasonic energy is delivered to where it is intended.

Clinical positioning devices and/or robots already exist and have been developed for other purposes, such as for performing robotic surgery, holding or delivering instruments and/or cameras, or aiding surgeons in general surgery. Such devices are able to precisely position and hold a tool for their intended purpose. Other tools, such as laparoscopes and endoscopes are also available, and used primarily for guidance and visualization.

### SUMMARY OF THE INVENTION

The present invention is a removable high-intensity focused ultrasound (HIFU) transducer and supporting system which includes an acoustically transparent and pliable vessel that is open at a top end and filled with acoustic coupling fluid, with the pliable vessel capable of holding a transducer assembly and a distal end of the positioning system within the pliable vessel as the HIFU treatment plan/process is executed. The vessel is fixed in place using a transducer and positioning system-independent external fixturing mechanism. The HIFU transducer and supporting system allows for HIFU transducer assemblies and other therapeutic ultrasound assemblies to be attached to third party positioning, imaging, or guidance systems while maintaining the required focal-zone positioning ability and the required acoustic energy coupling to tissue in order to mechanically or automatically ablate a target volume in unimpeded fashion.

The removable high-intensity focused ultrasound (HIFU) transducer and supporting system further includes an acoustically transparent, self-contained coupling structure which is placed between the face of the transducer assembly and the target tissue. The structure is attached to the transducer assembly and thus moves with the transducer assembly as the transducer assembly is re-positioned as the transducer assembly executes the HIFU treatment plan/process.

There is also provided an endoscope mounted transducer assembly which includes a high intensity focused ultrasound handpiece which has a channel for receiving an endoscope, and a first end which connects to a high intensity focused ultrasound transducer. The high intensity focused ultrasound transducer is removably connected, including O-rings or other attachment mechanisms. The high intensity focused ultrasound transducer is connected to an acoustic coupling which delivers energy, along with the endoscope, to a target tissue such as the tongue area, for treatment.

With the present invention, by adding a transducer to existing clinical positioning, monitoring devices, or guidance and visualization instrumentation, their utility can be extended, as they are now able to perform all functions required of a therapeutic ultrasound probe and system, pending that adequate tissue coupling can be achieved as well.

### BRIEF DESCRIPTION OF THE DRAWINGS

FIG. 1 is an illustration of the present invention.

FIG. 2 is an illustration of an embodiment of the present invention.

FIG. 3 is an illustration of an embodiment of the present invention.

FIG. 4 is an illustration of an embodiment of the present invention with a sample of a produced acoustic field.

FIGS. 5A and 5B are illustrations of the endoscope mounted transducer assembly of the present invention.

FIG. 6 is a driving system for powering the ultrasound transducer assembly.

FIG. 7 is an illustration of the present invention with a retractor blade.

FIG. 8 is an illustration of an embodiment of the present invention.

FIG. 9A is an illustration of the endoscope mounted removable transducer assembly of the present invention.

FIG. 9B is an illustration of a top view of the endoscope mounted removable transducer assembly of the present invention.

FIG. 9C is an illustration of the cone detail in cross section.

FIG. 9D is a front view of the endoscope assembly of the present invention.

FIG. 10 is an illustration of the endoscope mounted removable transducer assembly of the present invention.

FIGS. 11A-11D are endoscope mounted removable transducer assemblies of the present invention.

FIG. 12 is an illustration of the present invention in use during a medical procedure.

FIGS. 13A and 13B are illustrations of the present invention in use during medical procedures on the tongue base.

FIG. 14 is an illustration of the present invention in use during medical procedures on the tonsils.

FIG. 15 is an in vitro treatment with use of the present invention.

FIG. 16 is an in vitro treatment with use of the present invention.

FIG. 17 is an in vitro treatment with use of the present invention.

FIG. 18 is the controls and positioning system for use with the present invention.

### DETAILED DESCRIPTION

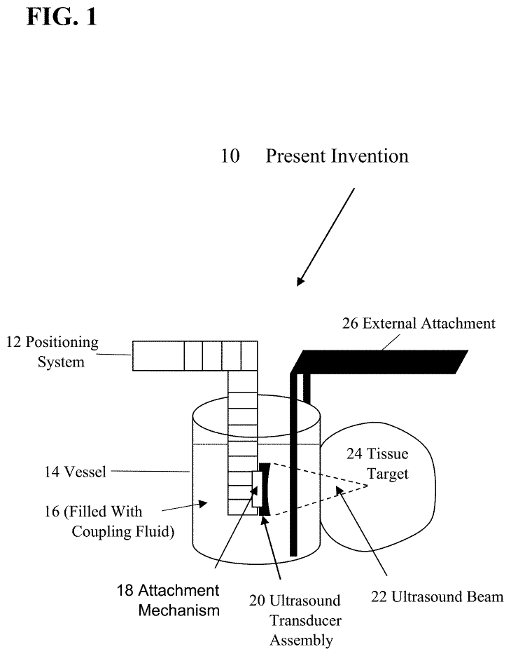

In FIG. 1, there is shown the present invention 10, which includes a positioning system 12 positioned into a vessel 14 that is filled with coupling fluid 16, an attachment mechanism 18 to connect the positioning system 12 with an high-intensity focused ultrasound transducer assembly 20. There is shown an ultrasound beam 22 produced by the ultrasound transducer assembly 20, with the ultrasound beam 22 delivering energy and being focused on a target tissue 24. The HIFU waves are concentrated from the transducer onto an ablation zone of the tissue during use of the present invention. An external attachment mechanism 26 is also shown to attach the present invention 10 at a fixed location. For the present invention 10, there is a self-contained ultrasound transducer assembly that is comprised of at least one therapeutic ultrasound transducer 20 (which can be focused, unfocused, high-intensity, array, etc.) and an attachment mechanism 18 (screw, clip, locking slide, etc.) that allows the assembly to be easily attached to the positioning system 12.

The vessel 14 is “sock-like” and can be opened on its top side, and is large enough to receive on its inside the transducer assembly 20 coupled to the positioning system 12 and is filled or can be filled with an ultrasound coupling fluid 16 (ie. water), and can also be held in place by an external attachment mechanism 26. This attachment mechanism 26 is not connected to the positioning system 12, but is a separate attachment mechanism which can be attached to or registered to the patient. The vessel 14 further allows positioning it so that it comes in contact with, and maintains its contact with the surface of the tissue 24 targeted for ultrasound exposure, is conformal/pliable, and the vessel 14 is made out of an acoustically and optionally optically transparent material (ie latex membrane, silicone membrane, or equivalent). The transducer assembly 20 coupled to the positioning system 12 are free to move within the vessel 14 to deliver the ultrasound beam 22, and are not negatively constrained by the shape of the vessel 14.

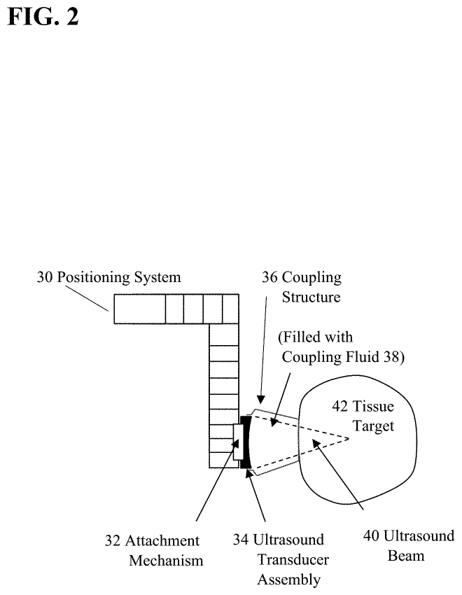

Referring to FIG. 2, there is shown a second embodiment of the present invention. There is shown a self-contained ultrasound transducer assembly as described with reference to FIG. 1 above. Again, there is a positioning system 30, an attachment mechanism 32, an ultrasound transducer assembly 34, a coupling structure 36 filled with coupling fluid 38. In FIG. 2, the ultrasound beam 40 is shown directed at the tissue target 42. In this embodiment, there is an acoustically transparent coupling structure 36 (“pillow-like”, or a full-fledged bolus assembly) that can be temporarily attached to the face of the ultrasound transducer assembly 34 so as to provide acoustic coupling between the ultrasound transducer assembly 34 and the target tissue 42. This structure 36 moves with the transducer assembly 34 while it is being moved by the positioning system 30, all the while maintaining the transducer assembly 34 acoustically coupled to the target tissue 42.

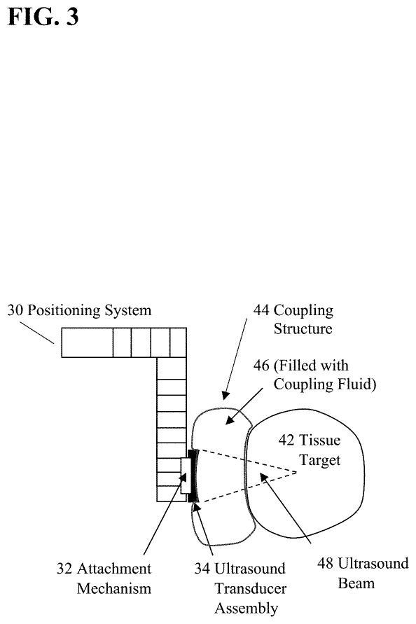

In FIG. 3, there is shown a third embodiment of the present invention. Again, there is provided a self-contained ultrasound transducer assembly as described in FIG. 1 above. An acoustically transparent coupling structure 44 that is “pillow-like” or a full-fledged bolus assembly, is positioned between the transducer assembly 34 and the target tissue 42, and is not tightly coupled to either transducer 34 nor tissue 42, which allows for continued coupling between the transducer assembly 34 and target tissue 42 as the transducer assembly 34 slides across its surface while delivering the ultrasound energy 40 to the target tissue 42. The coupling fluid 46 is contained in the coupling structure 44 as before.

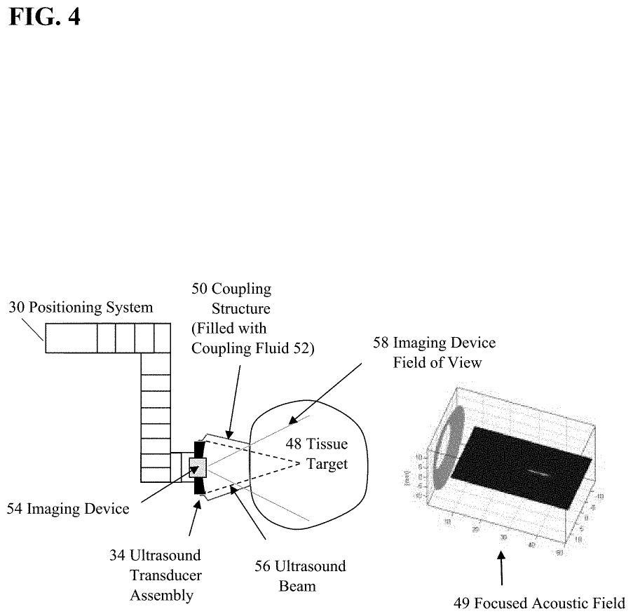

Referring now to FIG. 4, there is a fourth embodiment of the present invention. In this embodiment, the transducer assembly 34 is forward facing, and in the shape of a ring. This allows for concentric alignment between the transducer assembly 34 and possible other imaging devices 54 (video camera, laser, CCD, etc.) at the tip of the positioning system 30. The imaging device 54 is aligned with the transducer 34 in such a way that it peeks through a central opening of the transducer assembly 34. Mounting structures on its rear-facing aperture would attach the transducer assembly 34 to the tip of the positioning system. While any of the previously described coupling structures in FIGS. 1, 2, and 3 are compatible with this transducer and positioning system 30 arrangement, the coupling in FIG. 2 is preferred. In FIG. 4, there is shown the coupling structure 50 with coupling fluid 52 and imaging device field of view 58 from the position of the imaging device 54. The ultrasound beam 56 is directed at the tissue target 48 and there is also shown a typical focused acoustic field 49 such a transducer assembly 34 generates.

The following additional features and embodiments are within the scope of the present invention. The vessel and coupling structures (FIGS. 1, 2 and 3) may be single use and/or sterilizable. Similarly, the transducer assembly in FIG. 1 and FIG. 4 may also be single use and/or reusable, and may also be sterilizable. The transducer assembly of FIGS. 1 and 4 may incorporate an imaging transducer or array. Temperature sensor(s) may also be included. The ultrasound transducer delivering the ultrasound energy for therapy may be a single-element transducer or contain multiple elements (a phased array).

The invention described in FIG. 1, FIG. 2, or the invention described in FIG. 3 may incorporate a light source and video camera to help with visual positioning of the assembly on the target. The light source and/or the video camera must be registered and connected electronically or physically to the transducer assembly. The invention disclosed in FIG. 4 would have the video camera be concentrically aligned with the focal axis of the transducer assembly 34.

Vessel and coupling structure from FIGS. 1 and 3 may incorporate a frame or similar, that helps maintain the shape of the vessel. This frame may also serve as an attachment structure between the vessel 14 and its external attachment mechanism. The vessel and coupling structure(s) of FIGS. 1, 2 and 3 may incorporate fluid inlet and outlet ports to adjust the coupling fluid volume inside the structure, and a holding mechanism to support fluid inlet and outlet ports, so that the vessel may be filled and its fluid maintained (level, temperature, dissolved gas content, sterility, etc.) by an appropriate coupling fluid management system.

The vessel 14 and coupling structure(s) 36, 44, 50 may have external features (such as sleeves, slings, buttons, or equivalent), that allow it to be securely attached and held in place by its external attachment mechanism 26.

The coupling structures (FIGS. 2 and 3) may be pre-filled with an acoustically transparent medium (ie water), or may allow the user to fill it via build-in fluid inlet port, or may be primed, filled, and its volume maintained by an appropriate coupling fluid management system. The coupling structure 36 of FIG. 2 may also be in the shape of a cone and built using an acoustically transparent material that is solid and/or semi-solid (such as a gel or gelatine-like material). The base of the cone is in contact with the aperture of the transducer assembly 34 and follows its shape (ie a convex base shape that mates with the concave shape of the transducer 34, or a flat base shape that mates with the flat shape of the transducer), while the tip of the cone (likely a truncated tip) would be placed against the target tissue being treated.

The coupling structure 36 of FIG. 2 may also be in the form of an acoustic lens that may focus the ultrasound wave/beam 40 emanating from the transducer assembly 34. The coupling structure 44 of FIG. 3 may be in the shape of a cylinder, pillow, “sausage” or similar shapes.

The transducer assembly of FIG. 1 and FIG. 4 require at a minimum an ultrasound transducer amplifier/driving system to which it is connected. This system is able to generate the electrical signal(s) needed to power/excite the ultrasound transducer(s). This driving system also contains interfacing hardware and software that interfaces it to the positioning system (via a network, serial, USB, wife, or equivalent interface), so that the ultrasound transducer is being activated only when positioned/oriented at the desired location.

The invention described in FIG. 1 and FIG. 4 may be especially suitable for intracavity applications such as oral, rectal, vaginal, or minimal invasive or open surgical access. Additionally, the present invention described in FIG. 2, FIG. 3, or FIG. 4 may be especially suitable for intracavity applications such as oral, rectal, vaginal, or minimal invasive or open surgical access, or extracorporeal applications, in which the transducer assembly and coupling structure is in direct contact with the skin.

The attachment mechanism of FIG. 1 or FIG. 4 may be a clip, spring-loaded, screw, magnetic, slide-in, latch, or other mechanism. The external attachment mechanism 26 of vessel 14 may contain an open region/window, through which the ultrasound energy may propagate from the transducer to the target tissue without it being impeded by the attachment mechanism.

The acoustic coupling structure described in FIGS. 2 and 3 may be held in place by a vacuum mechanism that is part of the transducer assembly. When the vacuum is disengaged, the transducer assembly is able to move unimpeded with respect to the coupling structure. When the vacuum is engaged, the coupling structure 36 and 44 is “pulled in” and subsequently firmly attaches to the face of the transducer assembly, thereby ensuring good contact between the transducer's face (which is typically flat or concave) and the coupling structure. Ultrasound gel (or similar) may be further applied between the transducer assembly and any/all of the coupling structures described in this invention disclosure to (1) facilitate the movement of the transducer assembly 34 with respect to the coupling structure 36, 44, and (2) help facilitate the good coupling between the face of the transducer assembly and the coupling structure when the vacuum is engaged. Such a vacuum mechanism and corresponding vacuum port would likely be preferentially placed in the center of the aperture of the transducer assembly for best coupling results. Alternatively, several vacuum ports may be present, distributed on or around the aperture of the transducer

It should be noted that in any and all cases, the positioning system 12 mentioned can be as simple or as complex as desired by those of skill in the art. On the complex side of the range, such systems include motorized positioning assemblies, robotic manipulators, or manually activated positioning systems with XYZ positioning and rotation capabilities, etc. (“steppers”). On the simple side of the range, such systems could simply include a holding shaft to which the transducer assembly 20, 34, is coupled to, or could include other instruments, such as endoscopes or laryngoscopes. In these cases, the transducer assembly 20, 34 is simply attached to the endoscope, for example, so that the endoscope is able to function as such, but now also functions as a holding mechanism for the transducer assembly, in such a way that the endoscope/transducer assembly system can be manipulated as one instrument. This entire assembly is then coupled to the operating room table with a manipulating/articulating arm, such as those manufactured by Fisso.

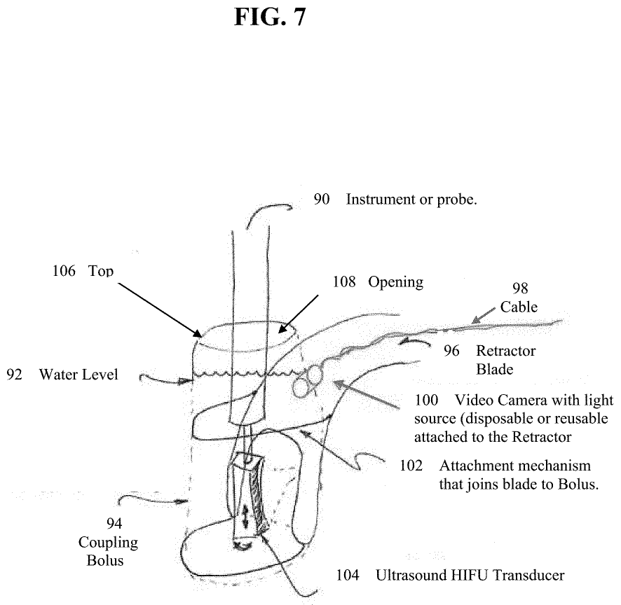

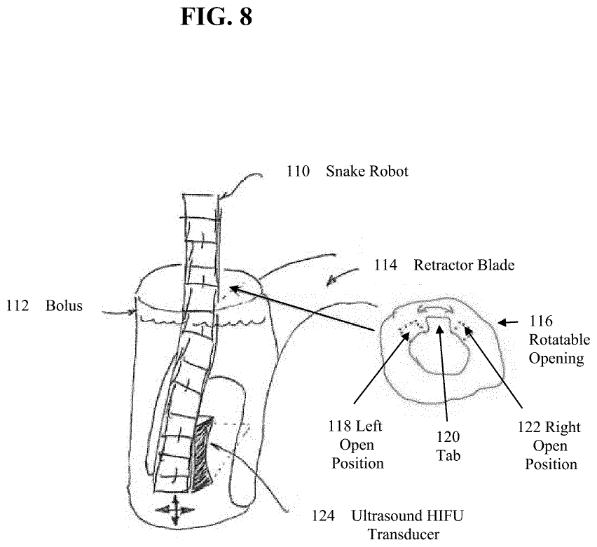

FIGS. 7 and 8 include additional features and embodiments of the present invention described with the previous Figures. In FIG. 7, there is shown an instrument or probe 90 laced through the opening 108 of the top 106 of a coupling bolus 94. The coupling bolus 94 is acoustically and optically transparent and the water level 92 inside the bolus is shown as indicated. The bolus is filled with degassed water and is closed at the bottom while open at the top for Sonatherm® or other instrument insertion. The bolus is also conformal a sit can be molded to the shape of the target, such as the base of the tongue. The water in the bolus provides ultrasound energy from coupling to the target. There is included a disposable and integrated retractor blade 96 for obstructive sleep apnea with the ultrasound coupling mechanism. A cable 98 with a video camera 100 having a light source (which may be disposable or reusable) is attached to the retractor blade 96. The retractor blade 96 is joined to the bolus 94 by attachment mechanism 102. At the bottom of the bolus 94 is the ultrasound HIFU transducer 104 attached to end of the probe 90, which disperses energy.

In FIG. 8, there is shown the use of a snake robot 110 which is inserted into the bolus 112 and the instrument with the ultrasound HIFU transducer 124 attached to the end of the snake robot 110. Retractor blade 114 is also included as before with FIG. 7. The snake robot 110 is then used to deliver the treatment of the HIFU directly to the tongue and target tissue. The bolus 112 is once again filled with degassed water, although other suitable liquids may be used. The top of the bolus 112 in this embodiment and others described herein can include a rotatable opening 116 with more than one position. The opening of the bolus can also be keyed. The ultrasound probe can then be inserted in a specific orientation and position. This will ensure the focal depth to be in the region of interest (ROI) and avoid user error. In addition, the opening 116 can be designed to rotate left and right from the center to allow for maximum lateral reach without interference of the beam by the two retractor blades and to ensure a controlled transmit based on the beam profile and not on the user. Here, three positions on the opening are shown: a left position 118, center tab 120 and a right position 122.

In FIG. 7, the transducer is free to move within the volume of the bolus 94, activated by the Sonatherm® probe. The water filled bolus provides acoustic energy coupling to the tongue base. In FIG. 8, however, the snake robot translates and positions the transducer up or down, and left versus right, allowing HIFU treatment of the tongue base.

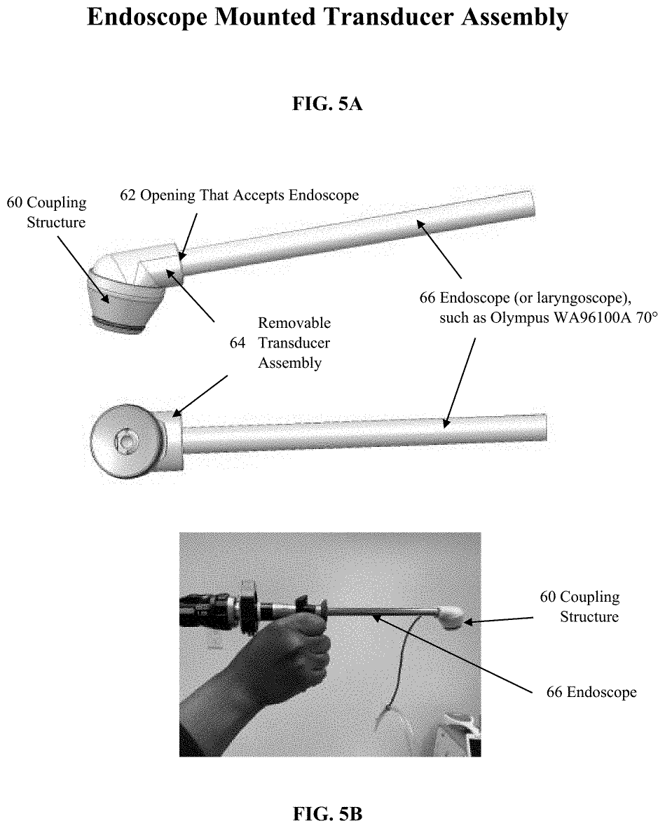

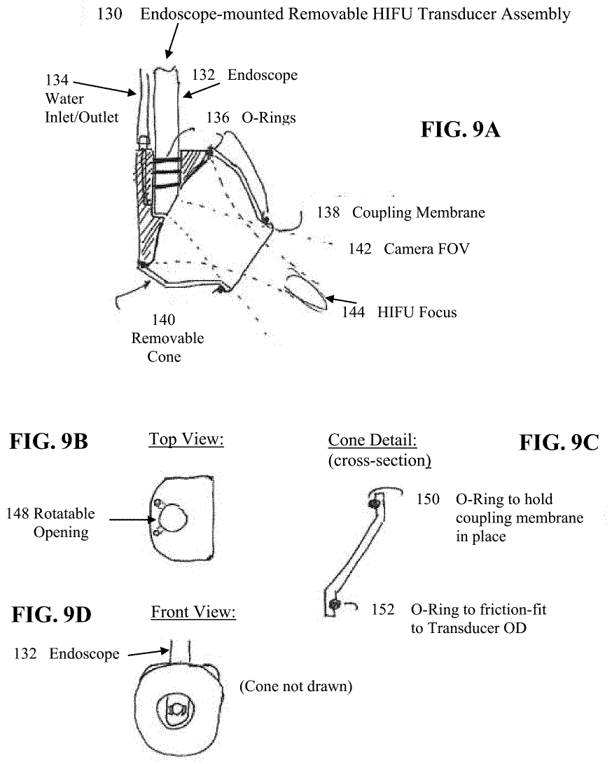

Referring to FIGS. 5A and 5B, there is another embodiment of the invention, illustrating an endoscope mounted transducer assembly. In the assembly, O-rings and mechanical clamping mechanisms are used to attach the transducer assembly to an endoscope, and is also shown in FIGS. 9A, 9B, 9C, and 9D. In FIG. 5A, there is shown the endoscope mounted transducer assembly, which includes coupling structure 60 with an opening 62 to accept the endoscope, removable transducer assembly 64, endoscope or laryngoscope 66, such as Olympus WA96100A 70 degrees. In FIG. 5B, there is shown the endoscope 66 and coupling structure 60.

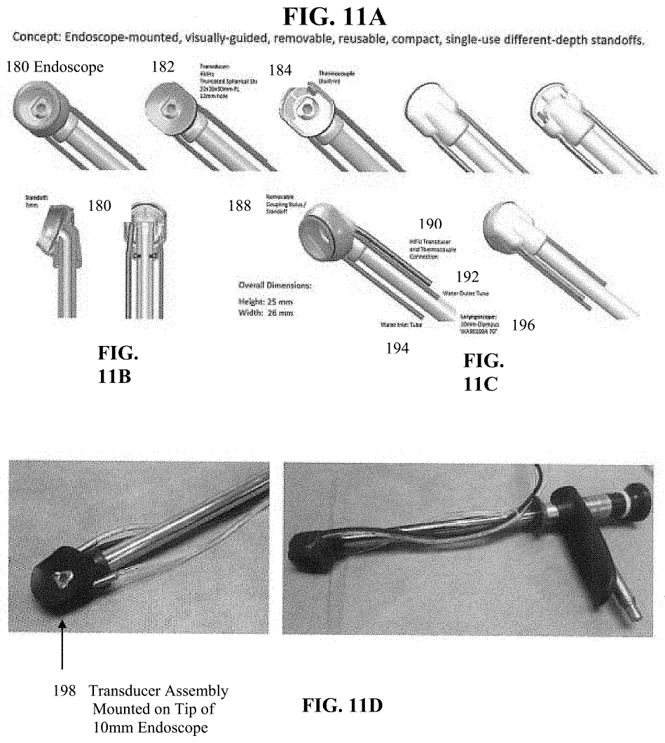

In FIG. 9A, there is shown the endoscope mounted removable HIFU assembly 130. The endoscope 132 with the parallel water inlet/outlet 134 is shown vertically. One or more O-rings 136 or similar mechanical attachment devices are used to secure the endoscope 132 with the transducer assembly. The camera field of view (FOV) 142 is indicated as extending form the removable cone 140 with the HIFU focus 144 of energy indicated. FIG. 9B is a top view, showing the rotatable opening 148. FIG. 9C is detail of the cone 140 in cross section and there is shown O-ring 150 to hold coupling membrane in place and O-ring 152 to friction-fit the cone to the transducer. FIG. 9D is a front view of the endoscope 132. The use of such an assembly is shown and described in FIGS. 11 to 14.

This design further contains a coupling structure (similar to that highlighted in embodiments described above), that consists of a solid cone 140 with an acoustically and optically transparent flexible membrane 138 at its distal end, and an O-ring based friction assembly 136 that allows this coupling cone 140 to be removably connected to the circumference of the HIFU transducer assembly. This allows the user to place different height cones onto the transducer, to allow for defining the treatment depth, based on the height of the cone. The distal end of the coupling cone 140 allows both the HIFU beam and the optical guidance beam to pass through, allowing optical placement of the assembly on the target. An example of how the coupling cone water inlet/outlet tubes 134 could be implemented is also shown. These continue to be required to fill the coupling cone 140 with fluid (water), to allow to couple the ultrasound energy 144 from the face of the HIFU transducer into the tissue target. The coupling cone 140 may be disposable.

Other attachment methods to the endoscope (other than the previously described friction-based O-ring structure) are also possible, as long as they meet certain requirements: (1) providing a sealed connection between the endoscope and transducer assembly to prevent the coupling fluid from escaping (if a cone-based coupling is used. Note that this requirement does not apply to a pillow or submersion-based coupling structure), (2) providing a reliable and solid coupling between both assemblies that solidly register the endoscope (and its optics) to the removable transducer assembly and its focal zone, and (3) provide an easy and simple way to attach and remove the transducer assembly to the endoscope by the clinician. If requirement (2) is not met, the utility of visualization through the coupling structure is greatly reduced, as it will not be useful for treatment planning and therapy guidance purposes as described in this disclosure. These include friction-based collars mounted around the distal tip of the endoscope that fit within the opening of the transducer assembly, and clip-on structures permanently mounted on the endoscope to which the transducer assembly snaps on/off, among other methods.

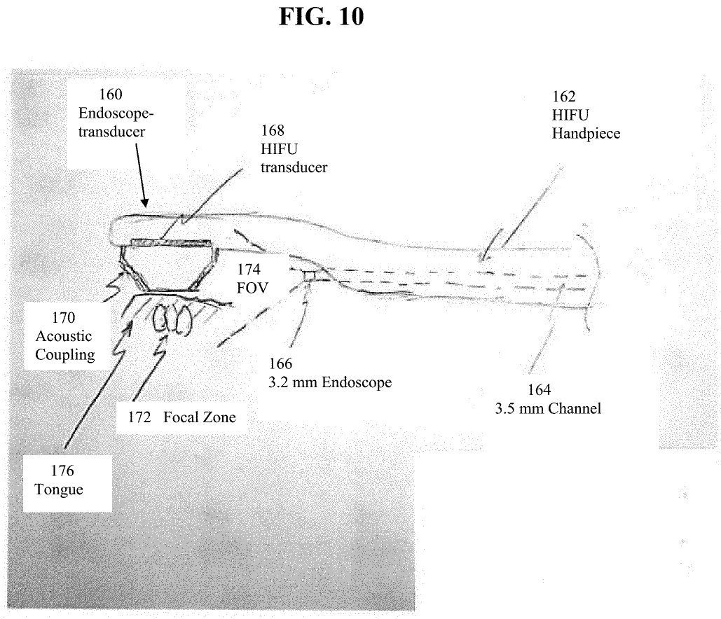

Endoscope attachment methods are also possible. While in the previous embodiments described above, the endoscope is aligned with the focal zone of the HIFU transducer and looks directly through the coupling fluid and front membrane of the coupling structure, the entire transducer assembly may also be attached to an endoscope positioned through its handle. This setup is shown in FIG. 10 where an endoscope mounted transducer assembly 160 is shown. There is also shown the HIFU handpiece 162 with a 3.5 mm channel 164 for a 3.2 mm endoscope 166, with the field of view 174 for the endoscope indicated. At the end of the handpiece 162 is the removably connected HIFU transducer 168 which is connected to the acoustic coupling 170. The acoustic coupling 170 is placed over the focal zone 172 of tongue 176 for treatment. Advantages of this setup include the ability to use smaller endoscopes, and being able to guide the positioning of the entire assembly from a field of view 174 that is external to the HIFU ablation zone 172.

Some other non-limiting specifics which may be used for these device embodiments described herein are as follows: The HIFU transducer aperture is 30 mm, with a focal length of 35 mm, and operating frequency of 4 MHz. The coupling cone height has different heights available with typical heights of: 15, 20, 25 mm (allowing for a treatment depth of 20, 15, and 10 mm, respectively). The endoscope: 10 mm diameter with angled, side-facing field of view (angled at 70 degrees), such as that implemented in the endoscope WA96100A from Olympus. The HIFU transducer is angled at the same angle, so that the endoscope's central part of its field of view is aligned with the focal zone of the HIFU transducer. Alternatively, the transducer assembly can be constructed in such a way as to accept a forward-facing endoscope, so that the transducer/endoscope combination is able to direct ultrasound energy forward, rather than at an angle. These dimensions allow the entire assembly to be used for oral applications, such as ablating the base of the tongue for the treatment of sleep apnea.

Alternatively, the following specifics in FIGS. 11A-D can also be used. The endoscope mounted is removable, re-usable and compact, with single use different depth standoffs. FIG. 11A illustrates an endoscope 180, a transducer 182, and a thermocouple 184. For FIG. 11B, there is shown the endoscope 180 and the standoff 186 of 7 mm. In FIG. 11C, there is the standoff 188, the HIFU 190, the water output 192 and the water input 194 and laryngoscope 196. The HIFU transducer aperture indicated is 22×26 mm (truncated spherical shell), focal length: 30 mm, operating frequency: 4 MHz. The coupling cone height: different heights available. Typical heights: 10, 15, 20 mm (allowing for a treatment depth of 20, 15, and 10 mm, respectively. An endoscope of 10 mm diameter with angled, side-facing field of view (angled at 70 degrees), such as that implemented in the endoscope WA96100A from Olympus. The HIFU transducer is angled at the same angle, so that the endoscope's central part of its field of view is aligned with the focal zone of the HIFU transducer. These dimensions generate an even smaller device, best suited for oral applications in combination with a mouth guard and tongue depressor, where insertion and device manipulation space in the oral cavity is very limited.

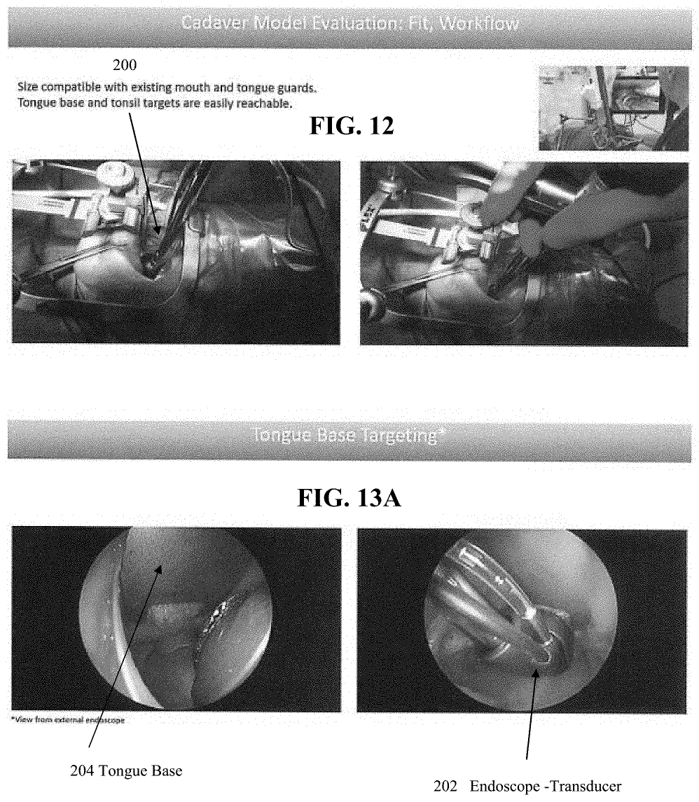

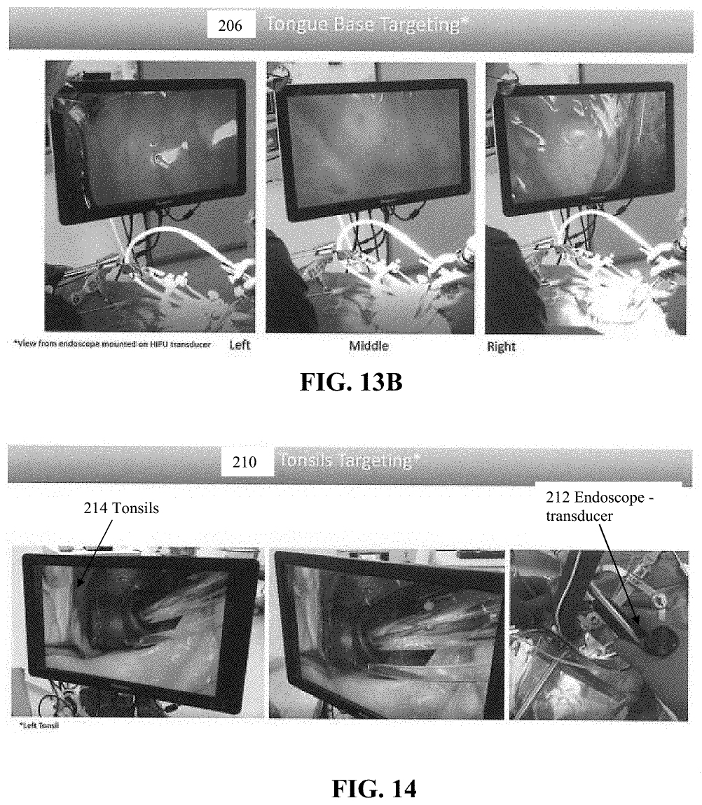

In FIGS. 12 to 14, there are demonstrated use of the endoscope devices from FIGS. 9A-D, 10 and 11A-D. FIG. 12 shows the use of the present invention 200 with the tongue base and tonsils as targets. FIG. 13A illustrates the present invention in use with the endoscope/transducer 202 shown targeting the tongue base 204. FIG. 13B illustrates the present invention targeting the left, middle, and right tongue base, showing the view from the endoscope mounted on the HIFU transducer. FIG. 14 is the present invention in use for tonsil targeting 210, with the endoscope mounted transducer 212 targeting tonsils 214.

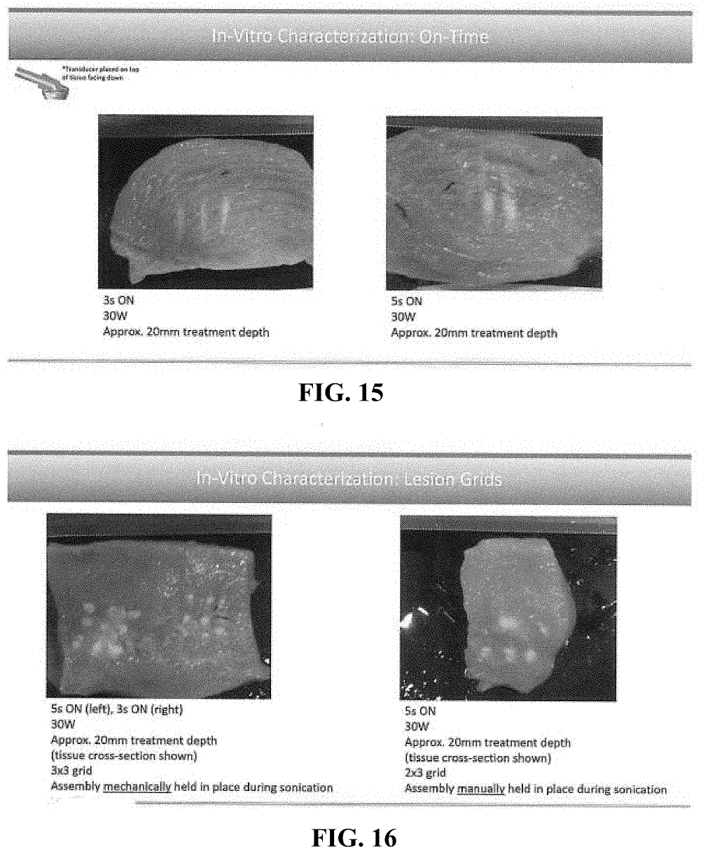

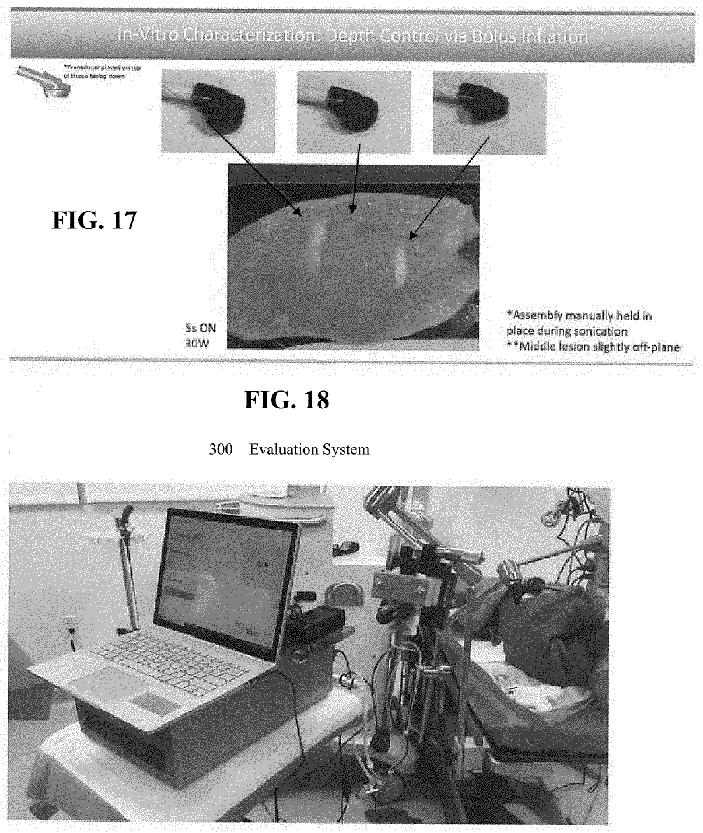

With reference to FIGS. 15-17, there are shown results of experiments with the present invention. In FIG. 15, there is an in vitro experiment where the transducer was placed on top of the target tissue facing down. There was 3 sec on and 5 sec on samples each at 30 watts with approximately 20 mm treatment depths. In FIG. 16, there is an in vitro experiment with lesion grids. In the left image, the invention was mechanically held in place and in the right image, the present invention was manually held in place during use. Approximately 20 mm treatment depths were made for each, both at 30 Watts. FIG. 17 is an in vitro experiment of depth control via bolus inflation. The transducer was placed on top of the tissue facing down and the assembly was manually held in place during sonication. The experiment was run at 5 sec on with 30 watts.

In FIG. 18, there is shown the set up for the use of the system 300 during a medical procedure. This includes the driving electronics and bolus control, the endoscope mounted transducer assembly, and a positioning system.

In another embodiment of the invention described in the prior figures, the coupling structure is flexible (inflatable/deflatable), but still visually transparent to be able to see through with the endoscope's field of view. Coupling fluid can be added/removed from this coupling structure, which results in decreasing or increasing the treatment depth. To exactly determine the treatment depth (which is now variable as the coupling structure is not rigid as in the previous instantiation), the HIFU transducer assembly itself is operated in pulse-echo mode, to determine the distance between the boundary of the pliable coupling structure (which is in contact with the target tissue) using time-of-flight calculations. This allows for a variable-treatment depth implementation of the concept, adding treatment flexibility. When the HIFU transducer is operated in pulse-echo mode, it can also be used to help monitor the delivery of the HIFU, by detecting, for example, the creation (or absence thereof) of vapor bubbles or acoustic impedance changes in the tissue in the focal zone of the transducer, brought about by the delivery of the HIDU energy.

Other imaging modalities can be used with this type of removable transducer assembly, all mounted in the central opening of the removable transducer assembly (instead of the previously described endoscope). These include gamma camera, gamma detector, infrared camera, an ultrasound imaging transducer, etc.

In yet another embodiment of the invention, there is a fingertip mounted transducer assembly. In this embodiment, the removable transducer assembly described thus far can also be mounted on a clinician's finger, and guided manually as well. The clinician simply inserts his/her finger into the hole where the endoscope would otherwise be mounted. This has the advantage of generating a very simple, manually-controlled ablation device, where the clinician uses his/her finger to position, hold, and translate the transducer assembly while delivering the therapy. Transducer coupling feedback is easily provided through such an arrangement. All other previously discussed features related to coupling structures (flexible, solid, differing heights, etc.) apply to this instantiation of the invention as well.

In usage of the present invention and work by medical professionals, the user attaches the transducer assembly to the positioning system (typically at the distal end of the positioning system's actuator), connects the transducer assembly to the driving system, and connects/interfaces the driving system to the positioning system.

For the invention described in FIG. 1, the user removes the coupling vessel from its packaging, attaches it to the attachment mechanism, and positions this next to/close to the target tissue that needs to be treated with ultrasound. At least one side of the vessel needs to be in contact with the target tissue, so that the ultrasound energy of the ultrasound transducer can be transmitted through the acoustic coupling media and then transmitted into the target tissue. The attachment mechanism is further attached to the patient, or registered to the patient. The vessel is subsequently filled with the coupling media using the coupling media management system through the appropriate ports/tubing. Note that the vessel is always aligned in such a way that its open section is facing upwards, to allow for containment of the coupling fluid/media. The distal end of the positioning system (with the ultrasound transducer assembly attached) is then positioned within the open end of the vessel in such a way that (i) the transducer assembly is fully submerged in the coupling fluid/media, (ii) the distal end of the actuator can freely move within the vessel, and (iii) the transducer is positioned so as to be able to deliver ultrasound energy to the target tissue. This can be done manually or by activating the positioning system's actuators. Optional: using ultrasonic and/or visual/optical guidance with the camera or other means, the positioning system's actuator position and orientation is fine-tuned by the operator to perfectly align the ultrasound transducer with the target tissue. Coupling to the tissue can be checked visually or acoustically.

For the invention described in FIG. 2 or the invention using an endoscope as the transducer's holding mechanism: The user removes the coupling structure from its packaging and attaches it to the ultrasound transducer assembly. Ultrasound gel (or similar) may be placed between the ultrasound transducer assembly and coupling structure to facilitate coupling. Ultrasound gel (or similar) may also be placed between the coupling structure and the target tissue. The vacuum (if this option is available/implemented) is activated to firmly attach the coupling structure to the transducer face. Coupling fluid is added/removed/adjusted, as needed through the coupling fluid inlet/outlet ports. The distal end of the positioning system or endoscope (with the transducer assembly and coupling structure attached) is then positioned so that the tip of the coupling structure comes in contact with the target tissue. This can be done manually or by activating the positioning system's actuators. Optional: using ultrasonic and/or visual/optical guidance with the camera or other means, the positioning system's actuator position and orientation is fine-tuned by the operator to perfectly align the ultrasound transducer with the target tissue. Coupling to the tissue can be checked visually or acoustically.

For the invention described in FIG. 3, the user removes the coupling structure from its packaging, and places it between the transducer assembly and the target tissue. Ultrasound gel (or similar) may be placed between the ultrasound transducer assembly and coupling structure to facilitate coupling. Ultrasound gel (or similar) may also be placed between the coupling structure and the target tissue. The vacuum (if this option is available/implemented) is activated to firmly attach the coupling structure to the transducer face. Coupling fluid is added/removed/adjusted, as needed through the coupling fluid inlet/outlet ports. Vacuum is now disengaged (if present), to allow for the distal end of the positioning system (with the transducer assembly attached) to be positioned so that the transducer assembly and the transducer's focal zone is targeting the desired tissue. Vacuum is now re-engaged (if present). This can be done manually or by activating the positioning system's actuators. Optional: using ultrasonic and/or visual/optical guidance with the camera or other means, the positioning system's actuator position and orientation is fine-tuned by the operator to perfectly align the ultrasound transducer with the target tissue. Coupling to the tissue can be checked visually or acoustically.

Once either system is correctly positioned, the user will define a treatment plan (typically consisting of a motion trajectory the positioning system will follow to translate the ultrasound transducer assembly along this trajectory, and an ultrasound dose), and execute the treatment. Alternatively, the treatment plan can consist of a simple collection of target points on the target tissue surface, and the transducer/endoscope assembly can be manually positioned on each of the target locations. During this phase, the positioning system will position the ultrasound transducer at desired points/trajectories, and the driving system will energize/excite the ultrasound transducer at the correct locations/timepoints to deliver the desired dose of ultrasound energy. If present, a vacuum will always be applied prior to ultrasound delivery (to assure good coupling between the transducer and the coupling structure) (such as for implementations of FIG. 2 and FIG. 3), but disengaged when the transducer assembly is being re-positioned for its next target zone (such as for the implementation in FIG. 3).

For the endoscope-mounted embodiments of the present invention, the user removes the coupling structure from its packaging, and places it on the transducer assembly (after having chosen the correct height based on the target depth). The endoscope is then inserted into the transducer assembly, holding the transducer assembly in place, and creating a self-contained endoscope-HIFU device. The bolus structure is now filled with coupling fluid (water) through the coupling structure's inlet/outlet ports, and all bubbles are removed from the circuit. Ultrasound gel (or similar) may now also be placed between the coupling structure and the target tissue. This structure is now positioned so that the transducer assembly and the transducer's focal zone is targeting the desired tissue, using the visual feedback from the endoscope as guidance for positioning the device, and the pulse/echo capability of the HIFU transducer for distance/depth measurement. The structure is then attached to the articulating arm and stepper/positioning assembly, and locked in place. Using ultrasonic and/or visual/optical guidance with the camera or other means, the positioning system's actuator position and orientation is fine-tuned by the operator to perfectly align the ultrasound transducer with the target tissue. Coupling fluid is added/removed based on the pulse-echo depth and desired treatment depth, to fine-tune the position of the focal zone within the target tissue. Coupling to the tissue can be checked visually or acoustically.

Once either system is correctly positioned, the user will define a treatment plan (typically consisting of a motion trajectory the positioning system will follow to translate the ultrasound transducer assembly along this trajectory, and an ultrasound dose), and execute the treatment. During this phase, the positioning system will position the ultrasound transducer at desired points/trajectories (or this will be accomplished manually by activating the mechanical stepper mechanism), and the driving system will energize/excite the ultrasound transducer at the correct locations/timepoints to deliver the desired dose of ultrasound energy.

The ideas described with this invention are specifically tailored to adding therapeutic ultrasound functionality/capability to existing clinical positioning systems or other clinical devices or positioning platforms that lack such functionality, and which may have been designed for other clinical applications initially. Examples include the Flex Robotic System such as those available from Medrobotics Corporation of Raynham, Mass., the Da Vinci surgical robot from Intuitive Surgical Inc. of Sunnyvale Calif., the endoscopes/laryngoscopes manufactured by Olympus, and similar clinical robotic/mechanical positioning systems or imaging subsystems (ultrasound, gamma, infrared, optical, etc.)

In effect, the positioning functionality and/or imaging functionality of the positioning/holding device is hijacked to provide positioning control and imaging guidance of the ultrasound transducer assembly. Their use can be extended by adding therapeutic ultrasound transducers as described. In such cases, the positioning devices are mainly used to control the position and orientation of the therapeutic ultrasound transducer assembly, allowing it to execute a treatment plan defined by a clinician, to deliver a specific dose of ultrasound energy to a tissue target volume.

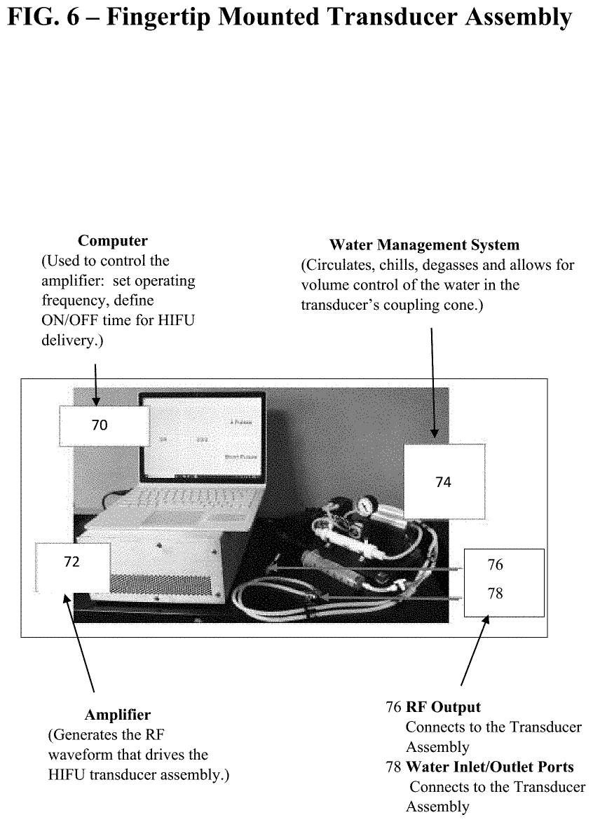

A separate device that powers the ultrasound transducer assembly is required, which further interfaces to the positioning device, so as to energize the ultrasound transducer only when the positioning system has positioned the ultrasound transducer assembly in its correct location and orientation to deliver the therapy. Such a device is implemented easily because its function is simply to energize/excite the ultrasound transducer, while other functions needed to deliver the ultrasound therapy (planning the treatment, motion control/translating the transducer to deliver the treatment at the desired location, etc.) are executed by the positioning system. The described vacuum functionality/control is part of this separate device as well. A simple tubing structure in this case connects the transducer assembly to this device. A picture showing the main components of such a driving system is shown in FIG. 6. In FIG. 6, there is shown a computer 70, an amplifier 72, a water management system 74, the RF output 76, and water inlet ports 78.

Clinical positioning system manufacturers or endoscope manufacturers not originally intending to develop an ultrasound-based therapeutic device can thus now expand their system's capability by adding therapeutic ultrasound functionality with the concepts described in this invention.

While the removability, compactness/size, and attachability to existing motion/holding and visualization devices of this transducer assembly and associated coupling structure(s) make it ideal for intra-oral applications (such as for ablating the base of the tongue in obstructive sleep apnea, soft palate ablation for snoring management, and tonsil ablation), the described concepts also allow for its use in other clinical applications.

These include its use in ablation of skin cancer (or the ablation of any other superficial cancer whose treatment would benefit from direct visualization prior to ablation), delivering ablative energy non-invasively to abnormalities of the cervix (cancer, cervicitis, etc.), rectal polyps, oral cancers, esophageal cancers, Barrett's esophagitis, etc. In all cases, the ability to expand the utility of existing positioning systems and/or visualization systems with a therapeutic capability by the addition of this removable transducer assembly and coupling structure will be beneficial.

What is claimed is:

1. A removable high-intensity focused ultrasound (HIFU) transducer and supporting system, comprising:
an acoustically transparent and pliable vessel that is open at a top end and filled with acoustic coupling fluid, said pliable vessel capable of holding a transducer assembly and a distal end of a positioning system within said pliable vessel as a HIFU treatment process is executed;
said vessel fixed in place using a transducer with said positioning system which is independent of an external fixturing mechanism;
said HIFU transducer and supporting system allowing for HIFU transducer assemblies and other therapeutic ultrasound assemblies to be attached to third party positioning, imaging or guidance systems while maintaining a required focal-zone positioning ability and a required acoustic energy coupling to target tissue in order to mechanically or automatically ablate a target volume in unimpeded fashion.

2. The removable high-intensity focused ultrasound (HIFU) transducer and supporting system of claim 1 wherein said system includes an acoustically transparent, self-contained coupling structure which is placed between a face of the transducer assembly and the target tissue; said structure attached to the transducer assembly and moving with the transducer assembly as the transducer assembly is re-positioned as the transducer assembly executes the HIFU treatment process.

3. An endoscope mounted transducer device comprising:
a high intensity focused ultrasound handpiece having a channel for receiving an endoscope, and a first end;
a high intensity focused ultrasound transducer removably connected to said handpiece at said first end;
said high intensity focused ultrasound transducer connected to an acoustic coupling which delivers energy to a target tissue for treatment.
